(19) United States
(12) Patent Application Publication
(10) Pub. No.: US 2004/0247664 A1
(43) Pub. Date: Dec. 9, 2004

(54) Gel capsules containing active ingredients and use thereof

(76) Inventors: Michael Dreja, Koeln (DE); Claudia Klink, Willich (DE); Andreas Buhl, Langenfeld (DE)

Correspondence Address: HENKEL CORPORATION, THE TRIAD, SUITE 200, 2200 RENAISSANCE BLVD., GULPH MILLS, PA, 19406, US

(21) Appl. No.: 10/855,918

(22) Filed: May 27, 2004

Related U.S. Application Data
(63) Continuation of application No. PCT/EP02/12737, filed on Nov. 14, 2002.

(30) Foreign Application Priority Data
Nov. 27, 2001 (DE) 101 57 755.9

(51) Int. Cl. A61K 9/48; A61K 9/64

(52) U.S. Cl. 424/452; 264/4.1

(57) ABSTRACT

A gel capsule charged with an active substance or component in the form of a matrix or storage system containing the active substance or component, the capsule having at least one oil phase containing the active substance or component in a gel matrix based on at least one block copolymer, the active substance or component itself being the oil phase or being dissolved in a carrier oil. Also, a process for making the gel capsules.

### CROSS REFERENCE TO RELATED APPLICATIONS

This application is a continuation under 35 U.S.C. § 365(c) and 35 U.S.C. § 120 of international application PCT/EP02/12737, filed on Nov. 14, 2002. This application also claims priority under 35 U.S.C. § 119 of DE 101 57 755.0, filed Nov. 27, 2001, which is incorporated herein by reference in its entirety.

### BACKGROUND OF THE INVENTION

This invention relates to a process for the production of gel capsules charged with active substance(s) and/or active component(s). The invention also relates to the capsule systems obtained by this process and to their use.

For many products, it is attractive for aesthetic reasons to add constituents or active components separately in demarcated form, for example in the form of capsules, beads, drops or as a second phase. In addition to their aesthetic advantages, these spatial demarcations often have stability and formulation advantages.

Active substances or active components, such as perfumes, perfume mixtures, perfume preparations, essential oils, perfume oils and care oils, dyes or pharmaceutical active principles, which are used in cosmetic and/or pharmaceutical products or in detergents, often lose their activity during storage or directly in use. Many of these substances also have inadequate stability for use or cause troublesome interactions with other product constituents.

Accordingly, there are advantages in using such substances with maximum effect under control and in the required place.

For this reason, active substances and/or active components such as, for example, perfumes, perfume mixtures, perfume preparations, care oils and antibacterial agents, are added to the products in spatially demarcated, protected form. Sensitive substances are often encapsulated in capsules differing in size, adsorbed onto suitable carrier materials or chemically modified. Their release can then be activated by a suitable mechanism, for example mechanically by shearing or by diffusion directly from the matrix material.

Accordingly, there is an ongoing search for systems suitable for use as encapsulation, delivery or carrier systems.

There are already numerous commercial delivery systems based on porous polymer particles or liposomes (for example Mikrosponges® from Advanced Polymer Systems or Nanotopes® from Ciba-Geigy, cf. B. Herzog, K. Sommer, W. Baschong, J. Röding: 37 Nanotopes™: A Surfactant Resistant Carrier System” in SÖFW-Journal, Vol. 124, October 1998, pages 614 to 623).

The disadvantage of these conventional delivery systems known from the prior art is that they only have a limited charging potential, the particle size of the polymer spheres is generally of the order of a few micrometers to a few 100 μm and the active substances generally cannot be encapsulated in situ. The capsule surfaces are either impossible or very difficult to modify. In addition, liposomes lack the stability required for many applications.

Another disadvantage of these conventional systems is that the release of the active substances at the place where they are specifically needed often cannot be controlled.

Another disadvantage of the conventional systems is that they do not allow a switch mechanism to be built into the capsules to control the release of the ingredients.

In addition, in the conventional production of capsules charged with active substance(s) and/or component(s), troublesome or toxic, foul-smelling or aggressive constituents are often introduced into the formulation. The encapsulation process is often carried out under aggressive conditions (high temperatures, long reaction times, occurrence of free radicals, etc.) which place the active components or active substances to be encapsulated under unnecessary stress.

Accordingly, one of the problems addressed by the present invention was to provide a capsule system in the form of a matrix or storage system containing active substances or active components, which would have improved properties in relation to the prior art, and a corresponding production process.

Another problem addressed by the present invention was in particular to develop capsules with a temperature switch or sustained-release capsules with a long-time effect for the release of active substances or active components such as, for example, perfumes, perfume mixtures, perfume preparations, care oils, vitamins, hydrophobic components, antibacterial components or other ingredients and a process for the production of such capsules. In particular, the process according to the invention would allow the production of capsules of a particular size with high active substance contents which would be distinguished from the prior art by their advantageous properties. The capsules would be produced in particular without a polymerization process, i.e. without the use of free radicals which could destroy active substances or active components.

In addition, the process according to the invention would have the advantage that it could be used for virtually any, in particular hydrophobic, active substance or active component. The resulting capsules would be stable, but would be able to release the ingredient completely. It would be of particular advantage if the ingredient could be released from the capsule over a prolonged period during the use of the product containing the capsule either under the effect of temperature or without the effect of temperature and without any mechanical action on the part of the user. In addition, the percentage content of auxiliary material (for example materials for forming the capsule structure) would be minimal.

### DESCRIPTION OF THE INVENTION

Accordingly, the present invention relates to a process for the production of gel capsules charged with active substance(s) and/or active component(s) in the form of matrix and/or storage systems containing active substance(s) and/or active component(s), characterized by the following process steps:

(a) preparing a mixture of an oil phase containing active substance(s) and/or active component(s) and at least one block copolymer;

(b) preparing a water and surfactant mixture;

(c) dispersing the mixtures prepared in (a) and (b), optionally with heating to temperatures above the gel-forming temperature of the dispersion formed;

(d) optionally preparing a pre-emulsion and/or macroemulsion from the dispersion prepared in (c) with heating above the gel-forming temperature of the pre-emulsion and/or macroemulsion,

(e) preparing a miniemulsion from the dispersion obtainable in (c) or from the pre-emulsion and/or macroemulsion optionally prepared in (d) with heating to temperatures above the gel-forming temperature of the emulsion formed;

(f) cooling the miniemulsion prepared in (e) below its gel-forming temperature so that gel capsules charged with active substance(s) and/or active component(s) in the form of matrix and/or storage systems containing active substance(s) and/or active component(s) are formed; and

(g) optionally removing the matrix and/or storage systems containing active substance(s) and/or active component(s) thus obtained.

In the process according to the invention, the mixture of the at least one block copolymer and the oil phase containing active substance(s) and/or active component(s) can be prepared by adding the block copolymer with heating to the oil phase containing active substance(s) and/or active component(s), more particularly while stirring, or vice versa. The heating temperature should be above the gel-forming temperature of the resulting mixture.

In the process according to the invention, either the active substance or the active component itself can form the oil phase or the active substance or active component can be dissolved in a carrier oil. In the latter case, the carrier oil phase may be selected in particular from the group of paraffin oils, isoparaffin oils, silicone oils, glycerides, triglycerides, naphthalene-containing oils, hydrocarbon-containing solvents and mixtures thereof. The carrier oil phase is preferably inert to the active substance and/or the active component and to the block copolymer. Inert means in particular that the carrier oil does not enter into any reaction with the active substance and/or the active component or block copolymer.

The ratio of active substance or active component used to carrier oil optionally used can vary within wide limits. Thus, a ratio by quantity or weight of active substance and/or active component to carrier oil of 1:99 to 99:1 is possible.

In the process according to the invention, the dispersion is prepared in step (c) by introducing the mixture of at least one block copolymer and an oil phase containing active substance(s) and/or active component(s) prepared in step (a) into the water and surfactant mixture prepared in step (b) or vice versa.

The optional emulsification of the dispersion obtained in step (c) in step (d) of the process according to the invention may be carried out by shearing.

The preparation of the miniemulsion in step (e) of the process according to the invention may also be carried out by shearing, for example in the form of ultrasonication, high-pressure homogenization or microfluidizer treatment.

In the process according to the invention, the formation of the miniemulsion in step (e) may be carried out under a homogenizing pressure of 50 bar to 30,000 bar and preferably under a homogenizing pressure of 300 bar to 2,500 bar. The formation of the miniemulsion may be carried out in particular over a period of 10 seconds to 2 hours and preferably over a period of 1 minute to 20 minutes, depending on the volume of the miniemulsion. The miniemulsion is generally not formed spontaneously, but through the input of energy in the form homogenization one or more times. The homogenization process has in particular a throughput which depends on the size of the homogenizer. The treatment time of each emulsion droplet is of the order of milliseconds.

In the process according to the invention, the cooling in step (f) leads to solidification of the oil droplets of the miniemulsion charged with active substance(s) and/or active component(s) to form gel capsules charged with active substance(s) and/or active component(s) in the form of matrix and/or storage systems which correspond in their mean particle size to the oil phase droplets of the miniemulsion.

The gel formation in step (f) of the process according to the invention takes place through physical interactions, more particularly physical network formation of the block copolymer molecules in the oil phase.

The miniemulsion used in the process according to the invention is a substantially aqueous emulsion stabilized by a surfactant—of the block copolymers and the oil phase containing active substance(s) and/or active component(s). The emulsion obtained in accordance with the invention preferably has a mean particle size of the emulsified oil phase droplets of about 10 nm to about 600 nm and more particularly about 20 nm to about 500 nm.

Miniemulsions are dispersions of an aqueous phase, an oil phase and optionally one or more surfactants where unusually small droplet sizes are achieved. In other words, miniemulsions may be regarded as aqueous dispersions of stable oil droplets with droplet sizes of about 10 to about 600 nm which are obtained by intensive shearing of a system containing oil, water, a surfactant and a hydrophobic component. In the present case, the hydrophobic components required for the production of stable miniemulsions are the block copolymer and/or the oil phase containing active substance(s) and/or active component(s) which generally have poor solubility in water. The hydrophobic component suppresses the mass exchange between the various oil droplets by osmotic forces (Ostwald ripening), although immediately after formation of the miniemulsion the dispersion is only critically stabilized in regard to inter-particle collisions and the droplets themselves can still increase further in size through further collisions and fusion. This effect can be suppressed or reduced by the gel formation of the oil droplets.

In contrast to microemulsions, which may generally be regarded as thermodynamically stable and optically transparent emulsions with droplet sizes of generally about 2 to at most about 50 nm, which are prepared by mixing water, oil, surfactant and optionally co-surfactant, miniemulsions may be regarded as kinetically stable and optically opaque to cloudy emulsions with droplet sizes of generally about 10 to about 600 nm which are prepared by mixing water, oil, surfactant and optionally a (another) hydrophobic component (for example even an oil) by relatively intensive shearing, the droplet size in the miniemulsion being determined in particular by the input of energy and by the nature and quantity of the individual components, more particularly the surfactants. In contrast to conventional emulsions, the droplet size distributions in miniemulsions are virtually monodisperse. In general, miniemulsions—in contrast to microemulsions—are critically stabilized, i.e. a quantity of surfactant just sufficient to stabilize the systems, more particularly less than 5% by weight, is generally required, whereas the amount of surfactant required for microemulsions is far greater, amounting to about 5 to 15% by weight. In addition, the interfacial tension in miniemulsions is distinctly higher than in microemulsions.

Further information on miniemulsions can be found in the article by K. Landfester, F. Tiarks, H.-P. Hentze, M. Antonietti “Polyaddition in miniemulsions: A new route to polymer dispersions” in Macromol. Chem. Phys. 201, 1-5 (2000), of which the content is included herein by reference. Reference is also made to the publication cited therein by E. D. Sudol and M. S. El-Aasser in: “Emulsion Polymerization and Emulsion Polymers”, P. A. Lovell, M. S. El-Aasser, Eds., Chichester 1997, p. 699, of which the content is also included herein by reference.

Accordingly, the miniemulsion used in accordance with the invention is first prepared in step (e) of the process according to the invention. The microemulsion is prepared in known manner, cf. the literature references already cited, namely the article by Landfester et al., the publication cited therein by Sudol et al. and WO 98/02466, DE 196 28 142 A1, DE 196 28 143 A1 and EP 818 471 A1, of which the entire contents are included herein by reference.

To prepare the miniemulsion, an aqueous pre-emulsion or macroemulsion containing the active substance(s) and/or active component(s), the block copolymer, the surfactant (surface-active substance) and water may first be prepared by simple methods known per se.

After the mixture has been homogenized and optionally converted into a pre-emulsion or macroemulsion, the macroemulsion formed in this way is converted in known manner into a so-called miniemulsion, a very stable form of emulsion, for example by subjecting the macroemulsion produced beforehand to treatment by ultrasonication, high-pressure homogenization or by a microfluidizer. The fine dispersion of the components is generally achieved by a high local energy input.

The mean droplet size of the disperse phase of the miniemulsion used in accordance with the invention may generally be determined on the principle of quasielastic dynamic light scattering where the so-called z-averaged droplet diameter of the unimodal analysis of the autocorrelation function is obtained. The particle size and particle size distribution of the emulsified droplets in the miniemulsion ultimately also determine the particle size and particle size distribution of the end products or gel capsules and largely correspond therewith. The particle size and monodispersity of the gel capsules obtained may also be characterized by dynamic light scattering.

The removal of the gel capsules charged with active substance(s) or active component(s) optionally carried out in step (g) of the process according to the invention may be effected by typical methods, more particularly by freeze-drying (lyophilization), evaporation of the dispersant, ultrafiltration, dialysis or spray drying under moderate conditions.

In the process according to the invention, process steps (a) to (e) may all be carried out at temperatures above the gel-forming temperature of the particular mixtures, dispersions and/or emulsions. In general, these temperatures may be in the range from 20 to 200° C. and are preferably in the range from 50 to 95° C.

In the context of the invention, gels are understood in particular to be organogels in the form of dimensionally stable, readily deformable, liquid-rich disperse systems of block copolymer(s) and oil phase(s). In the present case, these gels form quasi “sponge-like” structures of the block copolymer(s) as the gel former (gelator) or gelling agent and the oil phase containing active substance(s) and/or active components as the dispersant. These “sponge-like” structures consist of a physical network, i.e. they form an association through physical interactions. Gels have a yield point and, in particular, lend themselves to elastic and/or plastic deformation. Below a gel-forming temperature T_{gel}—also known as the gelation temperature—characteristic of the particular gel, the association of gelling agent and dispersant forms a gel-like structure (T<T_{gel}) whereas, at temperatures above the gel-forming temperature T_{gel} (T>T_{gel}), it becomes liquid. Gels can also be characterized by their elasticity modulus G′ and their loss modulus (dissipative modulus) G″. An association of gelling agent and dispersant forms a gel-like structure when the elasticity modulus is greater than or equal to the loss modulus (G′≧G″) at a given oscillation or measuring frequency, which is the case below the gel-forming temperature T_{gel}. Above the gel-forming temperature T_{gel}, the gel structure collapses and the loss modulus is greater than the elasticity modulus (G′<G″).

In the context of the present invention, gel capsules are not conventional capsules with core/shell structures, but rather an association—formed by physical interactions—of block copolymer(s) as the gelling agent and oil phase(s) containing active substance(s) or active component(s) as the dispersant which form a “sponge-like” structure in the form of discrete shell-free gel particles below their gel-forming temperature.

In a preferred embodiment of the present invention, the process according to the invention is carried out as follows:

First, the active substance or active component phase is formed by mixing the oil phase and the gel former. To this end, the active substance and/or active component (for example a perfume) is heated and an inert, miscible carrier oil is optionally added. A gel former, more particularly a hydrophobic gel former, preferably a block copolymer, which forms solid organogels with the oil phase below the particular gel-forming temperature (T_{gel}), is stirred into the resulting warm mixture. The resulting mixture is melted at a temperature above the gel-forming temperature T_{gel} of the resulting mixture and then emulsified with vigorous stirring into a water and surfactant mixture at the same temperature. The water and surfactant mixture is separately prepared. The crude emulsion formed from oil phase and block copolymer on the one hand and from water and surfactant on the other hand is converted into a miniemulsion using a high-pressure homogenizer under a pressure of 500 to 2,000 bar. This miniemulsion is distinguished by the fact that it is particularly stable to Ostwald ripening and has a largely uniform particle size distribution. Subsequent cooling of the miniemulsion to temperatures below the gel-forming temperature (T_{gel}) of the miniemulsion, preferably to room temperature, leads to solidification of the contents of the capsule or particle. To this end, the miniemulsion is left to cool while stirring to a temperature below the gel forming temperature T_{gel} which is preferably below room temperature. The gel former in the oil phase droplets gels the oil and forms rigid, gel-like capsules or particles with no shells. These particles have a size of 10 nm to 600 nm. The gel capsule or particle dispersion may then be further processed, for example applied to cleaning cloths or incorporated in a detergent or shampoo.

The active substance or active component used in the process according to the invention is an—in particular—oil-soluble, preferably hydrophobic active substance or active component. The active substance and/or active component may preferably be selected from the group of perfumes, perfume mixtures and perfume preparations; oils, such as essential oils, perfume oils, care oils and silicone oils; antioxidants and biologically active substances; oil-soluble vitamins and vitamin complexes; enzymes and enzymatic systems; cosmetically active substances; detersive substances; proteins and lipids; waxes and fats; foam inhibitors; redeposition inhibitors and color protectors; soil repellents; bleach activators and optical brighteners; amines; dyes, pigments and/or coloring substances; and mixtures of the compounds mentioned above.

In one particular embodiment, the active substances and/or active components used in accordance with the invention may be substantially insoluble in water or at least formulated to dissolve only sparingly in the aqueous phase. In such a case, less than 10%, preferably less than 5% and more particularly less than 1% of the active substances and/or active components used in accordance with the invention is soluble in the aqueous phase.

The content of active substance(s) and/or active component(s) in the miniemulsion prepared in step (e) may vary within wide limits. In general, it is 0.01% by weight to 50% by weight and preferably 2% by weight to 30% by weight. The content of block copolymer in the miniemulsion prepared in step (e) may also vary within wide limits and, in particular, is 0.01% by weight to 50% by weight and preferably 2% by weight to 20% by weight. If a carrier oil for the active substance and/or the active component is present, its content may also vary within wide limits and, in particular, is in the range from 1% by weight to 50% by weight and preferably in the range from 2% by weight to 30% by weight. The water content of the miniemulsion prepared in step (e) may also vary within wide limits and is generally 50% by weight to 99% by weight and preferably 70% by weight to 90% by weight. The content of surfactant(s) in the miniemulsion prepared in step (e) may also vary within wide limits and is in the range from 0.01% by weight to 10% by weight and preferably in the range from 0.5% by weight to 5% by weight.

The block copolymer used in the process according to the invention may be in particular a hydrophobic copolymer which forms a gel, preferably an organogel, with the oil phase containing active substance(s) and/or active component(s) below the corresponding gel-forming temperature. Accordingly, the block copolymer used in accordance with the invention is, in particular, a copolymer with oil-gelling properties.

The block copolymer used in the process according to the invention may be a polymer (A-B- . . . )ₙ (n=number of recurring units) consisting of at least two blocks or components A,B . . . , where at least one of the blocks is a hard block and at least one other of the blocks is a soft block. In other words, the blocks differ from one another in their hardness which is reflected in particular in their glass transition temperatures.

The glass transition temperatures of the hard and soft blocks of the block copolymer should differ by at least 50° C., more particularly by at least 60° C. and preferably by at least 70° C.

In one particular embodiment, the hard block may have a glass transition temperature T_{g(hard)} of >20° C., more particularly >50° C. and preferably >90° C. and the soft block may have a glass transition temperature T_{g(soft)} of ≦20° C., more particularly ≦0° C. and preferably ≦−45° C.

At least one block of the block copolymer used in the process according to the invention, preferably the hard block, should be oil-insoluble or only sparingly oil-soluble or at best moderately oil-soluble while at least one other block of the block copolymer, preferably the soft block, should be made oil-soluble. More particularly, at least one block of the block copolymer, preferably the hard block, should be less oil-soluble than at least one other block of the block copolymer, preferably the soft block.

The hard block of the block copolymer may preferably be selected from the group of polystyrenes, poly(meth)acrylates, polycarbonates, polyesters, polyanilines, poly-p-phenylenes, polysulfone ethers, polyacrylonitriles, polyamides, polyimides, polyethers, polyvinyl chlorides and mixtures thereof. The soft block of the block copolymer may preferably be selected from the group of rubbers, more particularly optionally substituted polyalkylenes, preferably polybutadienes, and mixtures of rubbers or polyalkylenes, such as polybutadiene/ethylene, polybutadiene/propylene, polyethylene/ethylenes; polyvinyl alcohols; polyalkylene glycols, such as polyethylene glycols and polypropylene glycols; polydimethoxysiloxanes; polyurethanes.

More particularly, the block copolymer may be selected from styrene/alkylene block copolymers where the (poly)alkylene block may also be a mixed block as previously described (for example polystyrene/polyethylene/polybutylene block copolymer). The block copolymer may preferably be a styrene/butadiene block copolymer, styrene/ethylene/butylene block copolymer, styrene/propylene block copolymer, styrene/butylene/propylene block copolymer or styrene/rubber block copolymer. The glass transition temperature T_{g(hard)} of the hard block of a preferred block copolymer according to the invention should be, in particular, about 100° C. (for example polystyrene hard block) while the glass transition temperature T_{g(soft)} of the soft block of a preferred block copolymer according to the invention should be, in particular, about −55° C. (for example rubber soft block, such as polyethylene/butylene soft block).

Block copolymers with multiarm blocks, so-called “star block copolymers”, may also be used.

The surfactant (surface-active substance) used in the process according to the invention for formulating the miniemulsion may be an ionic or nonionic surfactant.

If a cationic surfactant is used in the process according to the invention, it may be selected from the group of quaternary ammonium compounds, such as dimethyl distearyl ammonium chloride (CTMA-Cl); esterquats, more particularly quaternized fatty acid trialkanolamine ester salts; salts of long-chain primary amines of quaternary ammonium compounds, such as hexadecyl trimethyl ammonium chloride; cetrimonium chloride or lauryl dimethyl benzyl ammonium chloride.

However, if an anionic surfactant is used, it may be selected from the group of soaps; alkyl benzene-sulfonates; alkanesulfonates; olefin sulfonates; alkyl-ether sulfonates; glycerol ether sulfonates; □-methyl ester sulfonates; sulfofatty acids; alkyl sulfates; fatty alcohol ether sulfates; glycerol ether sulfates; fatty acid ether sulfates; hydroxy mixed ether sulfates; monoglyceride (ether) sulfates; fatty acid amide (ether) sulfates; mono- and dialkyl sulfosuccinates; mono- and dialkyl sulfosuccinamates; sulfotriglycerides; amide soaps; ether carboxylic acids and salts thereof; fatty acid isethionates; fatty acid sarcosinates; fatty acid taurides; N-acylamino acids, such as acyl lactylates, acyl tartrates, acyl glutamates and acyl aspartates; alkyl oligoglucoside sulfates; protein fatty acid condensates (particularly wheat-based vegetable products); alkyl—(ether) phosphates.

Where nonionic surfactants are used in the process according to the invention, this surfactant may be selected from the group of (i) nonpolymeric nonionic surfactants, such as alkoxylated, preferably ethoxylated, fatty alcohols, alkylphenols, fatty amines and fatty acid amides; alkoxylated triglycerides, mixed ethers and mixed formals; optionally partly oxidized alk(en)yl oligoglycosides; glucuronic acid derivatives; fatty acid-N-alkyl glucamides; protein hydrolyzates, more particularly alkyl-modified protein hydrolyzates, low molecular weight chitosan compounds; sugar esters; sorbitan esters; amine oxides; and (ii) polymeric nonionic surfactants, such as fatty alcohol polyglycol ethers; alkylphenol polyglycol ethers; fatty acid polyglycol esters; fatty acid amide polyglycol ethers; fatty amine polyglycol ethers; polyol fatty acid esters; polysorbates.

In general, the gel capsules obtainable by the process according to the invention have a content of active substance(s) and/or active component(s) of 95% by weight to 0.1% by weight. The content of block copolymer(s) is preferably from 5% by weight to 95% by weight. The content of carrier oil phase optionally present may be up to about 95% by weight. If a potent active component is used in a low concentration, the rest is made up by carrier oil, cf. the foregoing observations.

The present invention also relates to the gel capsules charged with active substance(s) and/or active component(s) in the form of matrix and/or storage systems containing active substance(s) and/or active component(s) obtainable by the process according to the invention.

The gel capsules charged with active substance(s) and/or active component(s) in the form of matrix and/or storage systems containing active substance(s) and/or active component(s) produced by the process according to the invention preferably contain at least one oil phase containing active substance(s) and/or active component(s) in a gel matrix based on at least one block copolymer. The active substance and/or the active component itself may form the oil phase or may be dissolved in a carrier oil, in which case the carrier oil phase may be selected from the group of paraffin oils, isoparaffin oils, silicone oils, glycerides, triglycerides, naphthalene-containing oils, hydrocarbon-containing solvents and mixtures thereof.

The ratio between the active substance or active component used in the gel capsules and the carrier oil optionally used may vary within wide limits. Thus, a ratio by quantity or by weight of active substance and/or active component to carrier oil of 1:99 to 99:1 is possible.

The gel matrix in the gel capsules according to the invention may be formed by physical interactions, more particularly by physical network formation, between the oil phase containing active substance(s) and/or active component(s) on the one hand and the at least one block copolymer on the other hand.

The gel capsules according to the invention charged with active substances or active components generally have a particle size of about 10 nm to about 600 nm and more particularly of about 20 nm to about 500 nm.

The gel capsules according to the invention of oil phase and block copolymer form a particulate, “sponge-like” structure of oil phase and block copolymer. In this structure, the oil phase and block copolymer may be present in homogeneous distribution, although the block copolymers are preferably present in associated form and the oil phase is distributed therein.

The gel capsules according to the invention charged with active substances or active components contain an—in particular—oil-soluble, preferably hydrophobic active substance or active component. The active substance or active component may be selected in particular from the group of perfumes, perfume mixtures, perfume preparations; oils, such as essential oils, perfume oils, care oils and silicone oils; antioxidants and biologically active substances; oil-soluble vitamins and vitamin complexes; enzymes and enzymatic systems; cosmetically active substances; detersive substances; proteins and lipids; waxes and fats; foam inhibitors; redeposition inhibitors and color protectors; soil repellents; bleach activators and optical brighteners; amines; dyes, pigments and/or coloring substances; and mixtures of the compounds mentioned above.

The active substance and/or active component present in the gel capsules according to the invention may be substantially insoluble in water or at least formulated to dissolve only sparingly in the aqueous phase, in which case generally less than 10%, preferably less than 5% and more particularly less than 1% of the active substance and/or active component is soluble in the aqueous phase.

The gel capsules according to the invention generally have a content of active substance(s) and/or active component(s) of 95% by weight to 0.1% by weight. The content of block copolymer(s) may be from 5% by weight to 95% by weight. If a carrier oil phase is present, its content may be up to about 95% by weight. All the percentages by weight mentioned are based on the gel capsules. If a potent active component is used in a low concentration, the rest is made up by carrier oil, cf. the foregoing observations.

So far as the chemical nature of the block copolymer is concerned, reference may be made to the foregoing observations.

The present invention also relates to the use of the gelatin capsules according to the invention.

The potential applications of the gel capsules containing active substances or active components produced by the process according to the invention are very numerous and extensive.

Thus, the gel capsules obtainable by the process according to the invention may be used as delivery systems, particularly in the field of cosmetics and body care (for example for deodorants, hair treatment preparations, shampoos, shower and bath gels, etc.), in pharmacology, in adhesives processing and/or in detergents (for example in dishwashing detergents, fabric softeners, detergents for washing at different temperatures, etc.).

More particularly, the gel capsules containing active substances or active components produced by the process according to the invention may be used as delivery systems for the controlled release of active substances or active components. The active substances or active components are released through the choice and/or quantity of the composition of the gel capsules. In the context of the invention, composition is understood in particular to mean the nature and/or quantity of the block copolymer or the nature and/or quantity of the oil phase containing active substance(s) and/or active component(s).

The release of the active substances and/or active components may be controlled in particular by controlling the glass transition temperatures of the polymer blocks of the block copolymer and hence through the gel softening temperature of the gel capsules.

The gel capsules according to the invention may be used in particular as delivery systems where the active substance and/or active component is dispensed over a relatively long period by prolonged or delayed release (sustained-release effect). More particularly, the active substance or active component is released without the application of external forces.

The present invention also relates to cosmetic preparations, body care preparations, pharmaceutical preparations, adhesives or detergents containing the gel capsules according to the invention in the form of matrix or storage systems containing active substance(s) or active component(s).

More particularly, the gel capsules charged with active substance(s) and/or active component(s) obtainable by the process according to the invention may be used in or on articles such as, preferably, cosmetic wipes or perfumed sheets (for example for use in tumble dryers), perfume strips or cards of board, card or paper and the like.

The present invention affords a number of advantages over the prior art.

In the process according to the invention, the gel structure is formed by physical interactions. Accordingly, the encapsulation process does not involve any polymerization steps, as is the case in the processes known from the prior art. Polymerizations where free radicals in particular are formed often lead to decomposition of the active substance and/or active component. Accordingly, the invention also provides an encapsulation process suitable even for sensitive active substances and/or active components.

In addition, the process according to the invention has the advantage that it may be used for virtually any, more particularly hydrophobic active substance and/or active component. The quantity ratio of active component(s) and/or active substance(s) to carrier oil phase optionally used is variable within wide limits. At the same time, a substantially monodisperse capsule size distribution is achieved by the miniemulsion process.

The gel capsules provided by the process according to the invention are smaller and more uniform particles than those obtained by known processes (droplet-forming, spray-drying or polymerization processes).

The gel capsules obtainable by the process according to the invention have an extremely high encapsulation efficiency. In general, active components and/or active substances may be encapsulated to a content of 95%. The open-pore gel capsule system allows a uniform and slow release of perfume which can be controlled through suitable compositions of the gel capsules.

In contrast to active substances and/or active components encapsulated in waxes or triglycerides, the gel capsules according to the invention contain far fewer residues. Since the gel capsules according to the invention do not have troublesome, insoluble capsule shells, problem-free further processing to many interesting products is possible. The many potential applications of the gel capsules according to the invention are also attributable to the broad range of variation of the hardness of the gel capsules.

Further embodiments, modifications and variations and also advantages of the present invention will become readily apparent to the expert on reading the description and practicable without having to depart from the scope of the invention.

As used herein, the articles “a” and “an” mean at least one or one or more, disclosing or encompassing both the singular and the plural, unless specifically defined otherwise. The conjunction “or” is used herein in its inclusive disjunctive sense, such that phrases formed by terms conjoined by “or” disclose or encompass each term alone as well as any combination of terms so conjoined, unless specifically defined otherwise. All numerical quantities are understood to be modified by the word “about,” unless specifically noted otherwise or unless an exact amount is needed to define the invention over the prior art. The following Examples are intended to illustrate the invention without limiting it in any way.

### EXAMPLES

### Example 1

1a)

Orange terpene is heated to 88° C., 20% Kraton G-1651 (styrene/rubber block copolymer from Kraton Polymers) is added and the whole is stirred at 88° C. to form a homogeneous mixture. The oil phase thus obtained (15 g) is dispersed into an aqueous phase of 150 g water and 3 g SDS (sodium dodecyl sulfate from Sigma) at a temperature of 95° C. using an Ultra-Turrax stirrer. The resulting crude emulsion is converted into a miniemulsion using a high-pressure homogenizer (5 mins. at a pressure of 1,000 bar). The miniemulsion is left to cool while stirring. Gelled orange oil particles with a particle size of 160 nm and a narrow particle size distribution are found.

The particle dispersion is applied to a cleaning cloth and releases a pleasant fragrance over a long period. After application of the cleaning cloths to a hard substrate (glass), the fragrance remains noticeable for a much longer time than is the case where unencapsulated, ungelled perfumes are used.

1b)

A gel phase was prepared under the same test conditions as in Example 1 using a mixture of orange terpene and Shellsol T (isoparaffin from Shell) in a ratio of 20:80.

The particle dispersion is applied to a cleaning cloth and releases a pleasant fragrance over a long period. After application of the cleaning cloths to a hard substrate (glass), the fragrance remains noticeable for a much longer time than is the case where unencapsulated, ungelled perfumes are used.

In addition, the presence of the detersive carrier oil gives the cloths a better cleaning effect against fatty soils.

### Example 2

A 5:95 mixture of rose oil and an inert isoparaffin carrier oil (Isopar M from Exxon) is heated to 84° C., 27% Kraton G-1650 (styrene/rubber block copolymer from Kraton Polymers) is added and the whole is stirred at 90° C. to form a homogeneous mixture. The oil phase (15 g) is dispersed into an aqueous phase of 150 g water and 2.7 g SDS (sodium dodecyl sulfate from Sigma) at a temperature of 90° C. using an Ultra-Turrax stirrer. The resulting crude emulsion is converted into a miniemulsion using a high-pressure homogenizer (5 mins. at a pressure of 1,000 bar). The miniemulsion is left to cool while stirring. Gelled rose oil/carrier oil particles with a particle size of 180 nm and a narrow particle size distribution are found. The particle dispersion is stirred into a fabric softener and, after application of the softener to laundry, releases a pleasant fragrance over a long period. After application, the fragrance remains noticeable for a much longer time than is the case where unencapsulated, ungelled perfumes are used.

### Example 3

A 5:95 mixture of vitamin A palmitate and an inert carrier oil (isopropyl palmitate) is heated to 82° C., 24% Versagel C HP (block copolymer from Penreco) is added and the whole is stirred at 80° C. to form a homogeneous mixture. The oil phase (15 g) is dispersed into an aqueous phase of 150 g water and 3 g DTAB (dodecyl trimethyl ammonium bromide, Aldrich) at a temperature of 80° C. using an Ultra-Turrax stirrer. The resulting crude emulsion is converted into a miniemulsion using a high-pressure homogenizer (3 mins. at a pressure of 900 bar). The miniemulsion is left to cool while stirring. Gelled vitamin A-in-carrier oil particles with a particle size of 120 nm and a narrow particle size distribution are found. The particle dispersion is stirred into a skin cream and contains the vitamin A palmitate stably in the product over a long period. Stability in storage is far higher than where unencapsulated, ungelled vitamin A palmitate is used.

What is claimed is:

1. A process for the production of gel capsules charged with an active substance or component in the form of a matrix or storage system comprising the active substance or component, comprising the steps of:
(a) forming a mixture of an oil phase comprising an active substance or component and a block copolymer;
(b) forming a water and surfactant mixture;
(c) forming a dispersion of the mixtures formed in (a) and (b), optionally with heating, at a temperature above a temperature at which the dispersion will form a gel;
(d) optionally forming a pre-emulsion or macroemulsion from the dispersion prepared in (c), optionally with heating, at a temperature above a temperature at which the pre-emulsion or macroemulsion will form a gel;
(e) forming a miniemulsion from the dispersion formed in (c) or from the pre-emulsion or macroemulsion optionally formed in (d), optionally with heating, at a temperature above a temperature at which the miniemulsion will form a gel;
(f) cooling the miniemulsion prepared in (e) to form gel capsules charged with the active substance or component, the capsules comprising a matrix or storage system comprising the active substance or component; and
(g) optionally separating gel capsules comprising the active substance or component from the miniemulsion.

2. The process of claim 1, wherein the mixture in step (a) is formed by combining the block copolymer and the oil phase containing active substance or component, with heating and optionally stirring, wherein the mixture temperature is above a temperature at which the mixture will form gel.

3. The process of claim 1, wherein the active substance or component itself comprises the oil phase or is dissolved in one or more carrier oils selected from the group consisting of paraffin oils, isoparaffin oils, silicone oils, glycerides, triglycerides, naphthalene-containing oils, hydrocarbon-containing solvents, and mixtures thereof.

4. The process of claim 1, wherein the dispersion in step (c) is formed by adding the mixture of the block copolymer and the oil phase comprising the active substance or component from step (a) to the water and surfactant mixture prepared in step (b), or vice versa.

5. The process of claim 1, wherein the optional emulsification in step (d) of the dispersion obtained in step (c) is carried out by shearing.

6. The process of claim 5, wherein the miniemulsion in step (e) is formed by shearing.

7. The process of claim 6, wherein the shearing comprises one or more of ultrasonication, high-pressure homogenization, or microfluidization.

8. The process of claim 1, wherein the miniemulsion in step (e) is formed under a homogenizing pressure of 50 bar to 30,000 bar.

9. The process of claim 8, wherein the homogenizing pressure is 300 bar to 2,500 bar.

10. The process of claim 1, wherein in step (f) oil droplets comprising the oil phase of the miniemulsion solidify to form gel capsules charged with the active substance or active component, the capsules having a mean particle size corresponding to the oil phase droplets of the miniemulsion.

11. The process of claim 1, wherein gel formation in step (f) takes place through physical network formation.

12. The process of claim 1, wherein the miniemulsion formed in (e) is a substantially aqueous emulsion, stabilized by the surfactant, of the block copolymer and the oil phase comprising the active substance or component.

13. The process of claim 12, wherein the oil phase comprises droplets having a mean particle size of 10 nm to 600 nm.

14. The process of claim 13, wherein the oil phase droplets have a mean particle size of 20 nm to 500 nm.

15. The process of claim 10, wherein the removal in step (g) is effected by freeze-drying, evaporation, ultrafiltration, dialysis, or spray-drying.

16. The process of claim 1, wherein process steps (a) to (e) are all carried out at temperatures above the gel-forming temperature of the particular mixture, dispersion, or emulsion.

17. The process of claim 1, wherein the active substance or component is oil-soluble.

18. The process of claim 17, wherein the active substance or component is hydrophobic.

19. The process of claim 1, wherein the active substance or component is selected from the group consisting of perfumes, perfume mixtures, perfume preparations, oils, essential oils, perfume oils, care oils, silicone oils, antioxidants, biologically active substances, oil-soluble vitamins, oil-soluble vitamin complexes, enzymes, enzymatic systems, cosmetically active substances, detersive substances, proteins, lipids, waxes, fats, foam inhibitors, redeposition inhibitors, color protectors, soil repellents, bleach activators, optical brighteners, amines, dyes, pigments, coloring substances, and mixtures thereof.

20. The process of claim 17, wherein the active substance or component is substantially insoluble in water.

21. The process of claim 17, wherein less than 10% by weight of the active substance or component dissolves in the aqueous phase.

22. The process of claim 21, wherein less than 5% by weight of the active substance or component dissolves in the aqueous phase.

23. The process of claim 22, wherein less than 1% by weight of the active substance or component dissolves in the aqueous phase.

24. The process of claim 3, wherein the miniemulsion prepared in step (e) comprises 0.01% by weight to 50% by weight of the active substance or component, 0.01% by weight to 50% by weight of the of block copolymer, 1% by weight to 50% by weight of the carrier oil or oils, 50% by weight to 99% by weight of water, and 0.01% by weight to 10% by weight of the surfactant or surfactants.

25. The process of claim 24, wherein the miniemulsion prepared in step (e) comprises 2% by weight to 30% by weight the active substance or component, 2% by weight to 20% by weight of the block copolymer, 2% by weight to 30% by weight of the carrier oil or oils, 70% by weight to 90% by weight of water, and 0.5% by weight to 5% by weight of the surfactant or surfactants.

26. The process of claim 1, wherein the block copolymer forms a gel with the oil phase.

27. The process of claim 26, wherein the block copolymer is a hydrophobic organic copolymer that forms an organogel with the oil phase.

28. The process of claim 26, wherein the block copolymer comprises at least two blocks or components, at least one of the blocks being a hard block and at least one other of the blocks being a soft block.

29. The process of claim 28, wherein the hard and soft blocks have glass transition temperatures that differ by at least 50° C.

30. The process of claim 29, wherein the hard and soft blocks have glass transition temperatures that differ by at least 60° C.

31. The process of claim 30, wherein the hard and soft blocks have glass transition temperatures that differ by at least 70° C.

32. The process of claim 28, wherein the hard block has a glass transition temperature T_{g(hard)} of >20° C. or the soft block has a glass transition temperature T_{g(soft)} of ≦20° C.

33. The process of claim 32, wherein the hard block has a glass transition temperature T_{g(hard)} of >50° C. or the soft block has a glass transition temperature T_{g(soft)}≦0° C.

34. The process of claim 33, wherein the hard block has a glass transition temperature T_{g(hard)} of >90° C. or the soft block has a glass transition temperature T_{g(soft)}≦−45° C.

35. The process of claim 28, wherein at least one block of the block copolymer is oil-insoluble or only sparingly oil-soluble and at least one other block of the block copolymer is oil-soluble.

36. The process of claim 28, wherein at least one block of the block copolymer is less oil-soluble than at least one other block of the block copolymer.

37. The process of claim 28, wherein the hard block of the block copolymer is selected from the group consisting of polystyrenes, poly(meth)acrylates, polycarbonates, polyesters, polyanilines, poly-p-phenylenes, polysulfone ethers, polyacrylonitriles, polyamides, polyimides, polyethers, polyvinyl chlorides, and mixtures thereof, or the soft block of the block copolymer is selected from the group consisting of rubbers, optionally substituted polyalkylenes, polybutadienes, mixtures of rubbers or polyalkylenes, polybutadiene/ethylene, polybutadiene/propylene, polyethylene/ethylenes, polyvinyl alcohols, polyalkylene glycols, polyethylene glycols, polypropylene glycols, polydimethoxysiloxanes, polyurethanes, and mixtures thereof.

38. The process of claim 37, wherein the block copolymer is a styrene/butadiene block copolymer, styrene/butylene block copolymer, styrene/propylene block copolymer, styrene/butylene/propylene block copolymer, or styrene/rubber block copolymer.

39. The process of claim 1, wherein the surfactant comprises one or more ionic or nonionic surfactants.

40. The process of claim 39, wherein the surfactant comprises a cationic surfactant selected from the group consisting of quaternary ammonium compounds, dimethyl distearyl ammonium chloride, esterquats, quaternized fatty acid triethanolamine ester salts, salts of long-chain primary amines of quaternary ammonium compounds, hexadecyl trimethyl ammonium chloride, cetrimonium chloride, lauryl dimethyl benzyl ammonium chloride, and mixtures thereof.

41. The process of claim 39, wherein the surfactant comprises one or more anionic surfactants selected from the group of consisting of soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, acyl lactylates, acyl tartrates, acyl glutamates, acyl aspartates, alkyl oligoglucoside sulfates, protein fatty acid condensates, alkyl (ether) phosphates, and mixtures thereof.

42. The process of claim 39, wherein the surfactant comprises one or more nonionic surfactants selected from the group consisting of nonpolymeric nonionic surfactants, alkoxylated fatty alcohols, alkylphenols, fatty amines, fatty acid amides, alkoxylated triglycerides, mixed ethers, mixed formals, optionally partly oxidized alk(en)yl oligoglycosides, glucuronic acid derivatives, fatty acid-N-alkyl glucamides, protein hydrolyzates, alkyl-modified protein hydrolyzates, low molecular weight chitosan compounds, sugar esters, sorbitan esters, amine oxides, polymeric nonionic surfactants, fatty alcohol polyglycol ethers, alkylphenol polyglycol ethers, fatty acid polyglycol esters, fatty acid amide polyglycol ethers, fatty amine polyglycol ethers, polyol fatty acid esters, polysorbates, and mixtures thereof.

43. The process of claim 3, wherein the gel capsules comprise 0.1% by weight to 95% by weight of the active substance or component, 5% by weight to 95% by weight of the block copolymer, and up to 95% by weight of the carrier oil.

44. A gel capsule charged with an active substance or component in the form of a matrix or storage system comprising the active substance or component, the capsule comprising at least one oil phase comprising the active substance or component in a gel matrix based on at least one block copolymer, the active substance or component itself being the oil phase or being dissolved in a carrier oil, the carrier oil being selected from the group consisting of paraffin oils, isoparaffin oils, silicone oils, glycerides, triglycerides, naphthalene-containing oils, hydrocarbon-containing solvents, and mixtures thereof.

45. The gel capsule of claim 44, having a particle size of about 10 nm to about 600 nm.

46. The gel capsule of claim 45, having a particle size of about 20 nm to about 500 nm.

47. The gel capsule of claim 44, in the form of a particulate structure of oil phase and block copolymer, the oil phase and block copolymer being present in homogeneous distribution or the block copolymers being present in associated form.

48. The gel capsule of claim 44, wherein the active substance or component is an oil-soluble substance or component selected from the group consisting of perfumes, perfume mixtures, perfume preparations, oils, essential oils, perfume oils, care oils, silicone oils, antioxidants, biologically active substances, oil-soluble vitamins, oil-soluble vitamin complexes, enzymes, enzymatic systems, cosmetically active substances, detersive substances, proteins, lipids, waxes, fats, foam inhibitors, redeposition inhibitors, color protectors, soil repellents, bleach activators, optical brighteners, amines, dyes, pigments, coloring substances, and mixtures thereof.

49. The gel capsule of claim 44, wherein the active substance or component is substantially insoluble in water.

50. The gel capsule of claim 44, comprising 0.1% by weight to 95% by weight of the active substance or component, 5% by weight to 95% by weight of the block copolymer, and up to 95% by weight of the carrier oil.

51. The gel capsule of claim 44, wherein the block copolymer forms a gel with the oil phase.

52. The gel capsule of claim 51, wherein the block copolymer is a hydrophobic organic copolymer that forms an organogel with the oil phase.

53. The gel capsule of claim 44, wherein the block copolymer comprises at least two blocks or components, at least one of the blocks being a hard block and at least one other of the blocks being a soft block.

54. The gel capsule of claim 53, wherein the hard and soft blocks have glass transition temperatures that differ by at least 50° C.

55. The gel capsule of claim 54, wherein the hard and soft blocks have glass transition temperatures that differ by at least 60° C.

56. The gel capsule of claim 55, wherein the hard and soft blocks have glass transition temperatures that differ by at least 70° C.

57. The gel capsule of claim 53, wherein the hard block has a glass transition temperature T_{g(hard)} of >20° C. or the soft block has a glass transition temperature T_{g(soft)} of ≦20° C.

58. The gel capsule of claim 57, wherein the hard block has a glass transition temperature T_{g(hard)} of >50° C. or the soft block has a glass transition temperature T_{g(soft)}≦0° C.

59. The gel capsule of claim 58, wherein the hard block has a glass transition temperature T_{g(hard)} of >90° C. or the soft block has a glass transition temperature T_{g(soft)}≦−45° C.

60. The process of claim 53, wherein at least one block of the block copolymer is oil-insoluble or only sparingly oil-soluble and at least one other block of the block copolymer is oil-soluble.

61. The gel capsule of claim 53, wherein at least one block of the block copolymer is less oil-soluble than at least one other block of the block copolymer.

62. The gel capsule of claim 53, wherein the hard block of the block copolymer is selected from the group consisting of polystyrenes, poly(meth)acrylates, polycarbonates, polyesters, polyanilines, poly-p-phenylenes, polysulfone ethers, polyacrylonitriles, polyamides, polyimides, polyethers, polyvinyl chlorides, and mixtures thereof, or the soft block of the block copolymer is selected from the group consisting of rubbers, optionally substituted polyalkylenes, polybutadienes, mixtures of rubbers or polyalkylenes, polybutadiene/ethylene, polybutadiene/propylene, polyethylene/ethylenes, polyvinyl alcohols, polyalkylene glycols, polyethylene glycols, polypropylene glycols, polydimethoxysiloxanes, polyurethanes, and mixtures thereof.
